(19) United States
(12) Patent Application Publication
(10) Pub. No.: US 2010/0106160 A1
(43) Pub. Date: Apr. 29, 2010

(54) INTRAOCULAR LENS INJECTION SYSTEMS AND METHODS

(76) Inventor: George Tsai, Mission Viejo, CA (US)

Correspondence Address: Knobbe, Martson, Olson & Bear LLP, 2040 Main Street, 14th Floor, Irvine, CA, 92614, US

(21) Appl. No.: 12/258,339

(22) Filed: Oct. 24, 2008

(51) Int. Cl. A61F 9/007

(52) U.S. Cl. 606/107

(57) ABSTRACT

An embodiment of an injector for an intraocular lens includes an injector body having a longitudinal axis. The body includes a first housing configured to receive the lens and a second housing configured to move relative to the first housing in the direction of the longitudinal axis. The injector also includes a lens engagement surface configured to engage a first, but not a second, viewing element of the lens. The injector includes opposing lens compaction members configured to move from a first position in which the lens compaction surfaces are spaced from each other to a second position in which the lens compaction surfaces are closer to each other and in which a lens positioned therebetween is compacted. In response to relative longitudinal movement of the housings, the lens engagement surface displaces the first viewing element from the second viewing element in the direction of the longitudinal axis, and the opposing lens compaction surfaces move from the first position to the second position. A retention member is configured to apply a longitudinal retention force on the lens compaction members to retain the compaction members in the second position.

### CROSS-REFERENCE TO RELATED APPLICATIONS

This application is related to U.S. patent application Ser. No. 11/046,154, filed Jan. 28, 2005, entitled “INJECTOR FOR INTRAOCULAR LENS SYSTEM,” which was published as U.S. Patent Application Publication No. 2005/0182419 on Aug. 18, 2005. The entire disclosures of the above-identified application and publication are hereby incorporated by reference herein and made a part of this specification.

### BACKGROUND

1. Field

Various embodiments disclosed herein pertain to insertion of an intraocular lens into an eye, as well as methods and devices for preparing an intraocular lens for insertion and for achieving the insertion itself.

2. Description of the Related Art

Artificial intraocular lenses are often implanted to replace the natural crystalline lens of an eye. Such a lens may be implanted where the natural lens has developed cataracts or has lost elasticity to create a condition of presbyopia. Devices have been developed to roll or fold an intraocular lens, and/or assist in implanting a rolled or folded lens through a small incision in the patient's eye. However, these known implantation devices suffer from various drawbacks, many of which are addressed by certain embodiments disclosed herein.

### SUMMARY

An embodiment of an injector for an intraocular lens that has first and second interconnected viewing elements with respective first and second viewing axes is disclosed. The injector comprises an injector body having a longitudinal axis. The body comprises a first housing and a second housing. The first and second housings are configured to move relative to each other in the direction of the longitudinal axis. The first housing is configured to receive the lens. The injector also comprises a lens engagement surface that is configured to engage the first, but not the second, viewing element. The injector also comprises opposing lens compaction members. In some variations, each lens compaction member comprises a lens compaction surface. The lens compaction members are configured to move from a first position in which the lens compaction surfaces are spaced from each other by a distance to a second position in which the lens compaction surfaces are closer to each other than in the first position and in which a lens positioned therebetween is fully compacted. In response to relative longitudinal movement of the housings, the lens engagement surface is configured to displace the first viewing element from the second viewing element in the direction of the longitudinal axis, and the opposing lens compaction surfaces move from the first position to the second position. The injector also comprises a retention member that is configured to apply a longitudinal retention force on the lens compaction members to retain the compaction members in the second position.

An embodiment of an intraocular lens injector for compacting an intraocular lens that has a first viewing element and a second viewing element is described. The injector comprises a housing that comprises a first portion and a second portion. The housing is configured to provide relative movement between the first and second portions. The housing has a first surface upon which the intraocular lens can be placed in an unstressed condition. The injector also comprises an injection lumen that has a projection extending at least partially in the housing. The projection of the injection lumen has a longitudinal axis. The injector also comprises a lens displacement member disposed within the housing opposite of the first surface. The lens displacement member is movable from a first displacement position relative to the first surface to a second displacement position. The injector also comprises a first lens compacting surface and at least one movable compacting member that comprises a second lens compacting surface. In some arrangements, the second lens compacting surface is disposed opposite the first lens compacting surface. The movable compacting member has a first compacting position in which at least one of the first and second lens compacting surfaces is spaced away from the projection of the injection lumen and has a second compacting position in which the first and second lens compacting surfaces are spaced substantially along the projection of the injection lumen. The injector also comprises a retention member positioned between the movable compacting member and the housing. The retention member is configured to bias the movable compacting member toward the second compacting position.

An embodiment of an injector for an intraocular lens is disclosed. The injector comprises a delivery lumen extending along a delivery axis and a lens compactor that has a home configuration for retaining the lens in a substantially unstressed condition and a compacted configuration in which the compactor stresses the lens into an at least partially compacted condition. The lens compactor is configured to change from the home configuration to the compacted configuration in response to movement of a compactor actuator by a user. The injector also comprises a driving member that is movable at least partially along the delivery axis and is configured to drive the lens along the delivery lumen when the lens is in the at least partially compacted state. The injector also comprises a locking member that has a locked position in which the driving member is substantially restricted from driving the lens when the compactor is in the home position and an unlocked position in which the driving member is substantially unrestricted from driving the lens when the compactor is in the compacted condition. The locking member may be changed from the locked position to the unlocked position in response to the movement of the compactor actuator by the user.

An embodiment of a method of preparing for implantation an intraocular lens having first and second interconnected viewing elements with respective first and second viewing axes is provided. The method comprises providing the intraocular lens along a longitudinal axis of a chamber such that the first and the second viewing axes are substantially colinear. The method also comprises relatively displacing the viewing elements along the longitudinal axis such that the viewing axes are no longer colinear and moving at least one compaction member to a compacted position in which the lens is at least partially compacted while remaining along the longitudinal axis. The method also comprises applying a retention force on the at least one compaction member in order to at least partially retain the at least one member in the compacted position.

### BRIEF DESCRIPTION OF THE DRAWINGS

FIG. 1A is an isometric view schematically illustrating an embodiment of an injector adapted to house an intraocular lens (not shown). The injector is depicted in an “open” position before the intraocular lens has been compacted.

FIG. 1B is an isometric view schematically illustrating the injector shown in FIG. 1A in a “closed” position in which first and second housings have been moved toward each other, and in response, the intraocular lens has been compacted.

FIG. 1C is an isometric view schematically illustrating the injector of FIGS. 1A and 1B in a “delivery” position after a plunger has been advanced to force the compacted intraocular lens (not shown) out of the injector.

FIGS. 2A-2C are top cross-sectional views of an embodiment of an injector at various stages during compaction of an embodiment of an intraocular lens. FIG. 2A schematically illustrates the injector in the open position. FIG. 2B schematically illustrates the injector in a “displaced” position in which two viewing elements of the intraocular lens are relatively displaced along a longitudinal axis of the injector. FIG. 2C schematically illustrates the injector in the closed position, in which the (previously displaced) intraocular lens has been compacted.

FIGS. 3A and 3B are side cross-sectional views of the injector, which correspond to the open and displaced positions shown in FIGS. 2A and 2B, respectively.

FIGS. 4A-4C are front views of the injector, which correspond to the open, displaced, and closed positions shown in FIGS. 2A-2C, respectively. The intraocular lens is not shown in FIGS. 4A-4C.

FIG. 5 is an exploded view schematically illustrating an embodiment of an injector.

FIGS. 6A-6C are exploded and perspective views that schematically illustrate movement of a lens engagement member that is configured to longitudinally displace viewing elements of the intraocular lens.

FIGS. 7A-7C are perspective views that schematically illustrate displacement of the intraocular lens by the lens engagement member (FIGS. 7A and 7B) and compaction of the intraocular lens by wedge-shaped lens compaction members (FIG. 7C).

FIG. 8A is a perspective view that schematically illustrates an embodiment of a mechanism for displacing and compacting an intraocular lens.

FIG. 8B is an exploded view of the mechanism in FIG. 8A.

FIG. 8C is a bottom perspective view of an embodiment of a carrier for a lens engagement member.

FIGS. 9A and 9B are closeup cutaway views schematically illustrating engagement of the distal ends of the lens compaction members with an injection lumen in the nozzle of an embodiment of an injector.

FIGS. 10A and 10B are cross-section views that schematically illustrate an embodiment of a plunger lock mechanism. The plunger is locked in FIG. 10A and unlocked in FIG. 10B.

FIGS. 10C and 10D are close-up views of the plunger lock mechanism illustrated in FIGS. 10A and 10B, respectively.

FIG. 11 is a flowchart that schematically illustrates an embodiment of a method for preparing an intraocular lens for implantation into an eye.

### DETAILED DESCRIPTION OF PREFERRED EMBODIMENTS

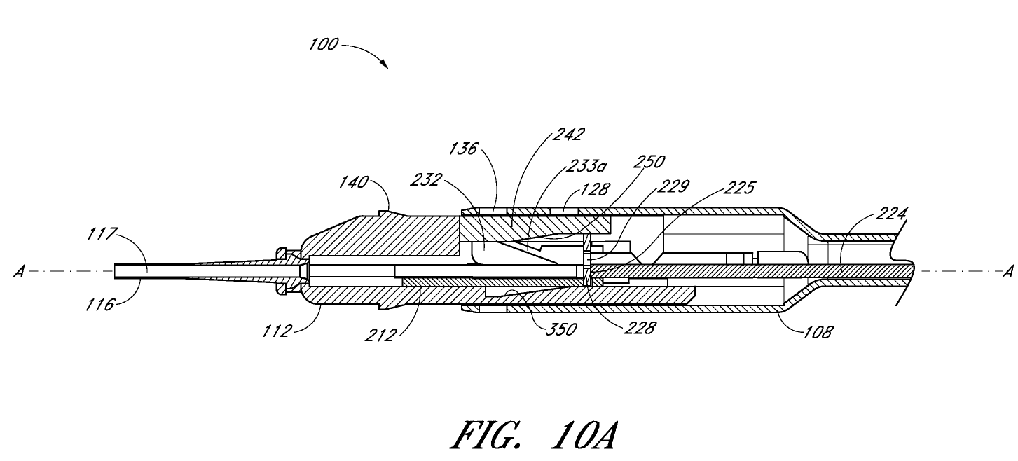

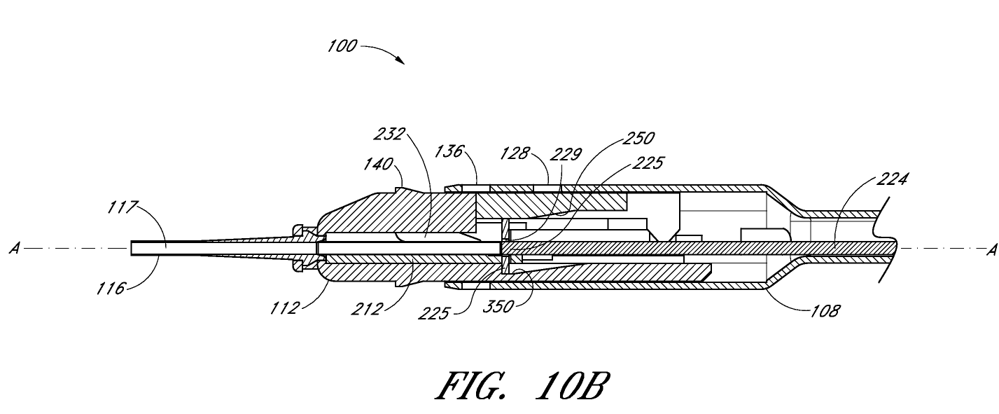

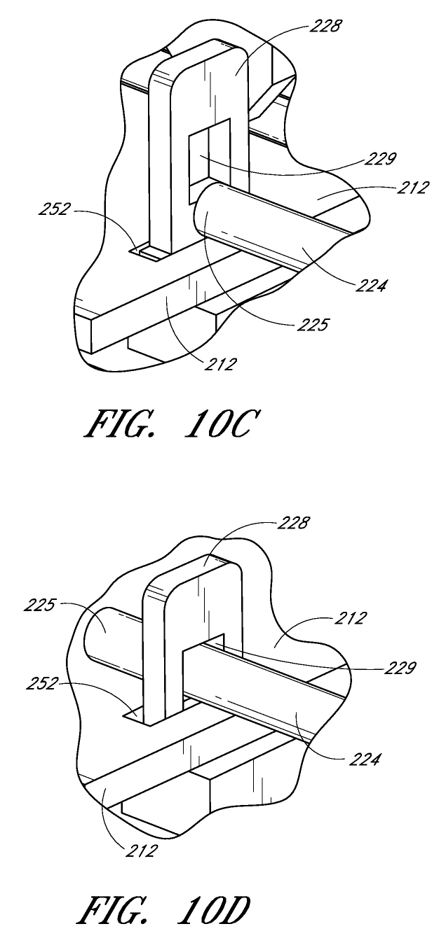

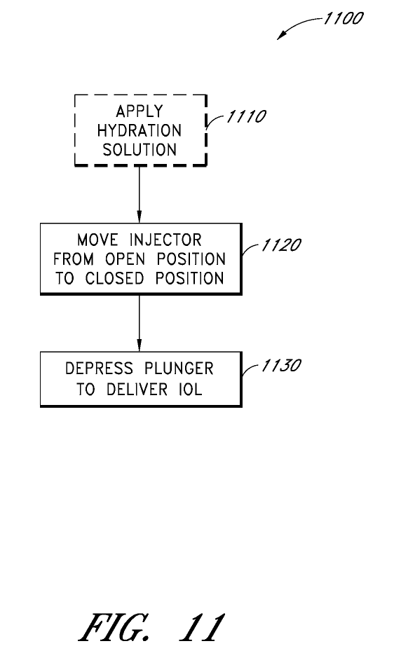

FIGS. 10A-10B schematically illustrate an embodiment of an injector 100 for injecting an intraocular lens (“IOL”) into a human or animal eye. Intraocular lenses may be implanted (typically after removal of the natural lens) by first compacting the IOL. The compacted IOL is then inserted into the desired location in the eye by passing the IOL through one or more incisions made in the cornea, sclera, and/or ciliary capsule. Once in place, the natural resilience of the IOL causes it to return, either partially or completely, to its original uncompacted state, whereupon the IOL can function as desired to improve the patient's vision. In certain advantageous embodiments, the injector 100 may be used to first compact an IOL and then to deliver the compacted IOL to the desired location in the eye.

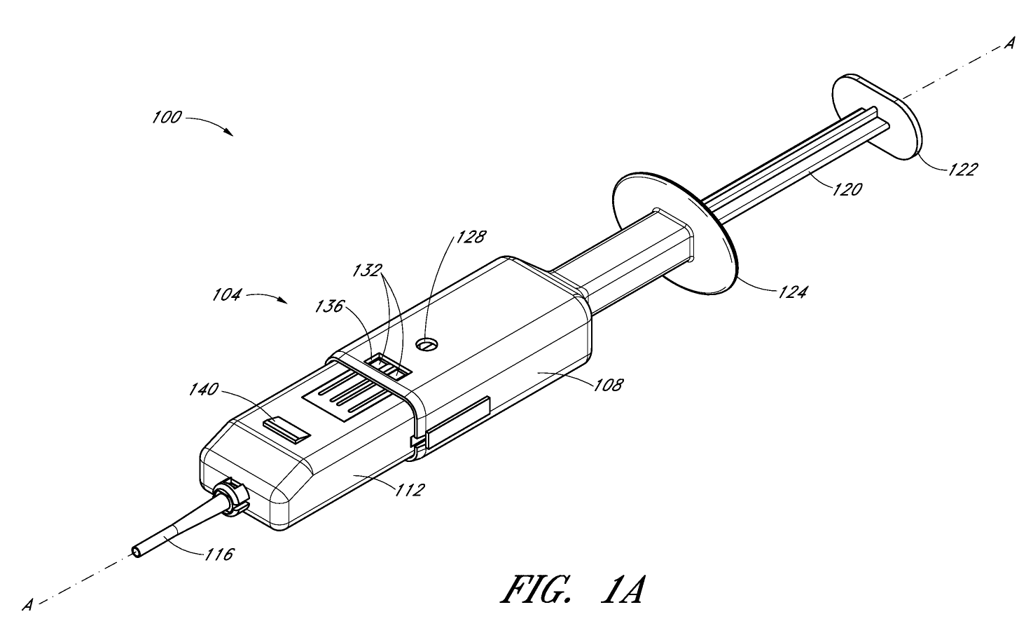

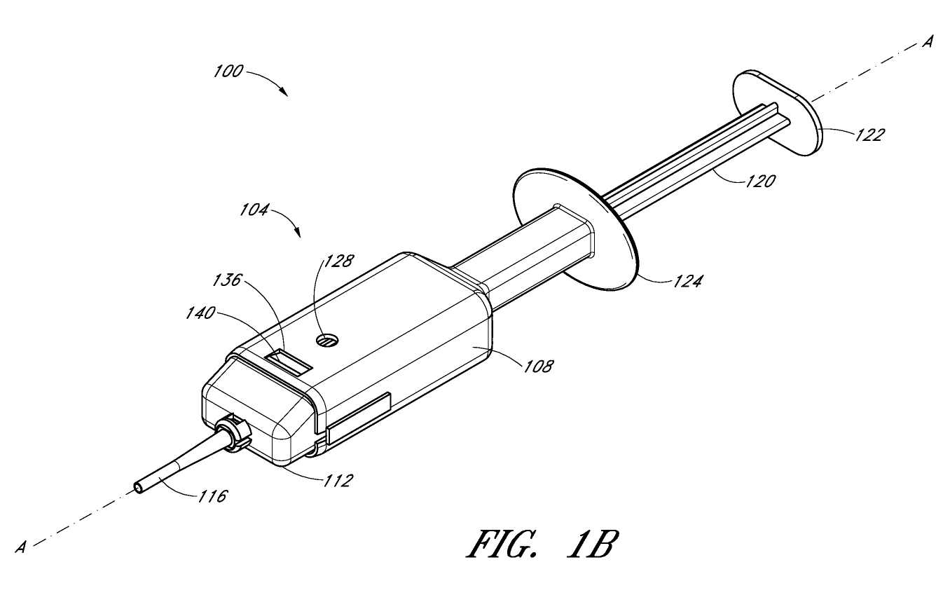

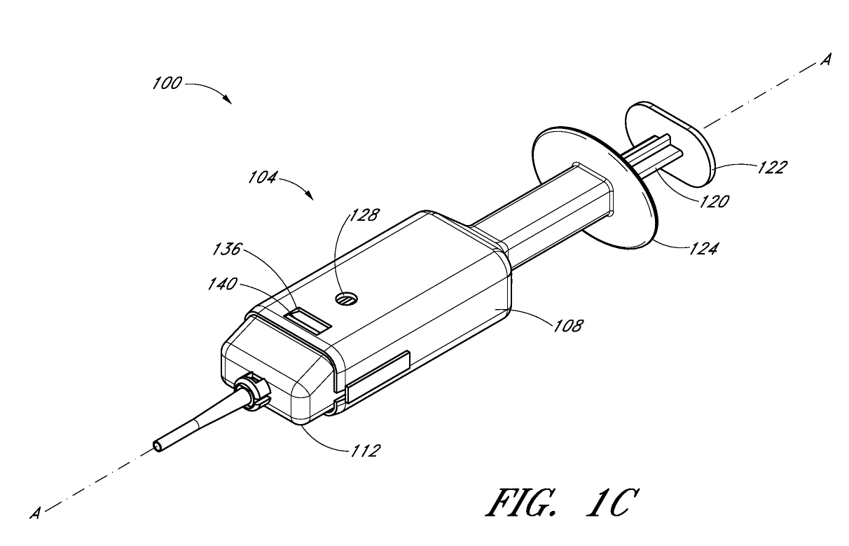

FIGS. 1A-1C are isometric views schematically illustrating an embodiment of the injector 100. The injector 100 comprises an injector body 104 having a longitudinal axis A-A. The injector body 104 comprises a first housing 108 and a second housing 112. The first housing 108 is configured to hold or store an IOL (not shown in FIG. 1A). The second housing 112 comprises an injection nozzle 116. The first housing 108 and the second housing 112 are movable relative to each other in a direction along the longitudinal axis A-A. In describing the injector 100, the terms “distal” or “forward” are used to describe directions longitudinally toward the injector nozzle 116, and the terms “proximal” or “rearward” are used to describe directions longitudinally away from the injector nozzle (e.g., towards a plunger 120). Also, the terms “transverse” and “vertical” are used to describe mutually orthogonal directions that are each orthogonal to the longitudinal axis A-A.

FIG. 1A schematically illustrates the injector 100 in an “open” position, before the first and the second housings 108, 112 are moved toward each other. In the open position, the IOL can be stored in the first housing 108. In one arrangement, the injector 100 is arranged such that the IOL can be stored therein in a substantially unstressed storage configuration or position. The open position may sometimes be referred to as a “home” position. The injector 100 may be moved to a “closed” position (schematically illustrated in FIG. 1B), for example, by grasping one of the housings 108, 112 and relatively moving the other of the housings 108, 112 along the longitudinal axis A-A toward the “grasped” housing. As will be further described below, the relative movement of the housings 108, 112 causes the IOL to be compacted within the injector 100. In this embodiment of the injector 100, the IOL remains substantially on the longitudinal axis A-A during compaction. The compacted IOL may be delivered into the patient's eye by moving a plunger 120 along the longitudinal axis A-A toward the injection nozzle 116. Movement of the plunger 120 displaces the compacted IOL along the longitudinal axis A-A and through an injection lumen 117 in the injection nozzle 116. FIG. IC schematically illustrates the injector 100 in a “delivery” position in which the plunger 120 has been fully depressed to eject the compacted IOL. The plunger 120 can be moved, for example, by placing a thumb against a thumb plate 122 on a proximal end of the plunger 120, placing fingers against a finger grip 124 on a proximal end of the first housing 108, and squeezing the thumb toward the fingers.

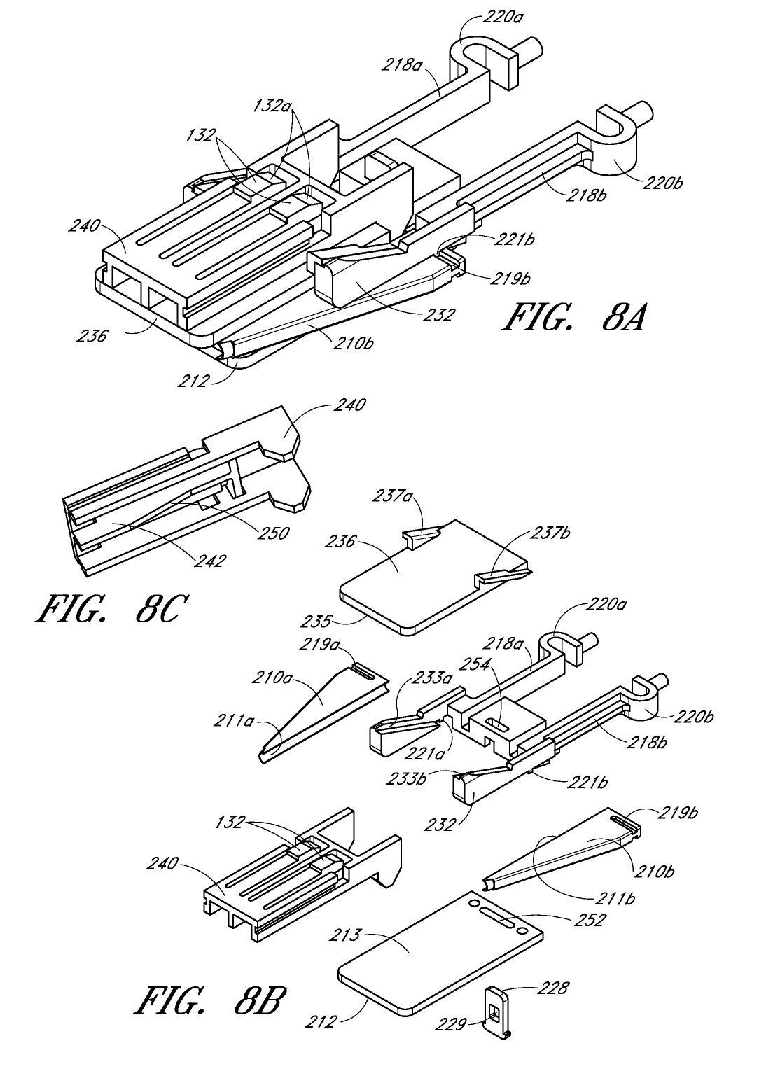

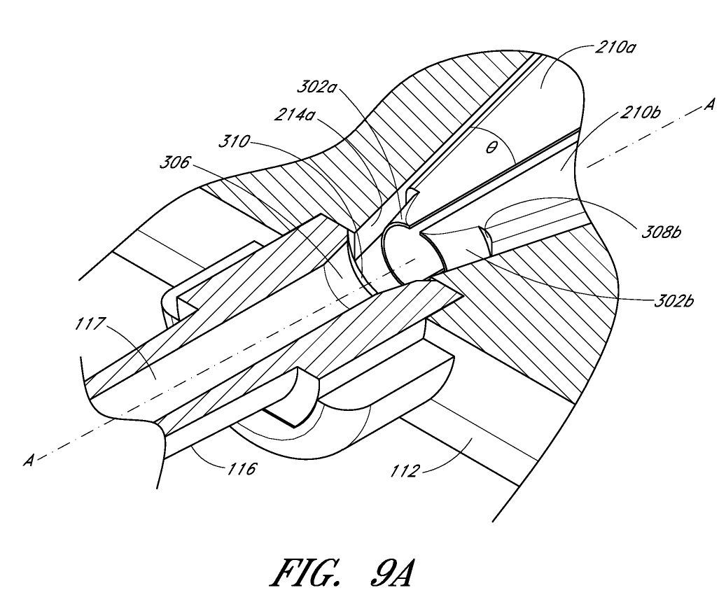

In the embodiment shown in FIGS. 1A-1C, the injector 100 has features that permit the injector to be “locked” into the open position or the closed position. For example, the second housing 112 comprises an engagement element 132 and a locking ramp 140, and the first housing 108 comprises an engagement slot 136. When the injector 100 is in the open position (see, e.g., FIG. 1A), a proximal end of the engagement element 132 engages the engagement slot 136, thereby preventing inadvertent relative movement of the housings 108, 112, which could cause undesired compaction of the IOL prior to an ophthalmic procedure. When a medical practitioner desires to move the injector 100 from the open position to the closed position, the practitioner may apply a force tending to urge the housings 108, 112 toward each other and which permits the engagement element 132 to disengage from the engagement slot 136. For example, as can be seen in FIG. 8A, the engagement element 132 may comprise ramped protrusions which permit the engagement element 132 to slide under an upper portion of the first housing 108 when the practitioner applies the force. The practitioner may also lightly depress the engagement element 136, e.g., along a vertical direction or axis, to permit the element to slide under the upper portion of the housing 108. As the injector 100 moves toward the closed position (see, e.g., FIG. 1B), the locking ramp 140 on the second housing 112 engages the engagement slot 136 in the first housing 108, thereby locking the housings 108, 112 into the closed position. Advantageously, this prevents the housings 108, 112 from moving apart along the longitudinal axis A-A, which could allow the compacted IOL to return, at least partially, to an uncompacted state.

In some embodiments, some or all of the movable portions of the injector 100 may be coated with a lubricious substance to reduce friction. Because some lubricious substances are activated by hydration, the first housing 108 may comprise a port 128 through which a hydrating solution may be administered (e.g., when the injector 100 is in the open position). The hydrating solution advantageously may be isotonic to eye tissue and may comprise, for example, water, saline, or balanced salt solution (BSS). The hydrating solution may be added before the injector 100 is moved from the open position (see, e.g., FIG. 1A) to the closed position (see, e.g., FIG. 1B). In some embodiments, the hydrating solution may provide some degree of lubrication. Various embodiments of lubricious coatings are discussed, for example, in U.S. patent application Ser. No. 11/046,154, filed Jan. 28, 2005, entitled “INJECTOR FOR INTRAOCULAR LENS SYSTEM,” which was published as U.S. Patent Application Publication No. 2005/0182419 on Aug. 18, 2005. The entire disclosures of the above-identified application and publication are hereby incorporated by reference herein and made a part of this specification.

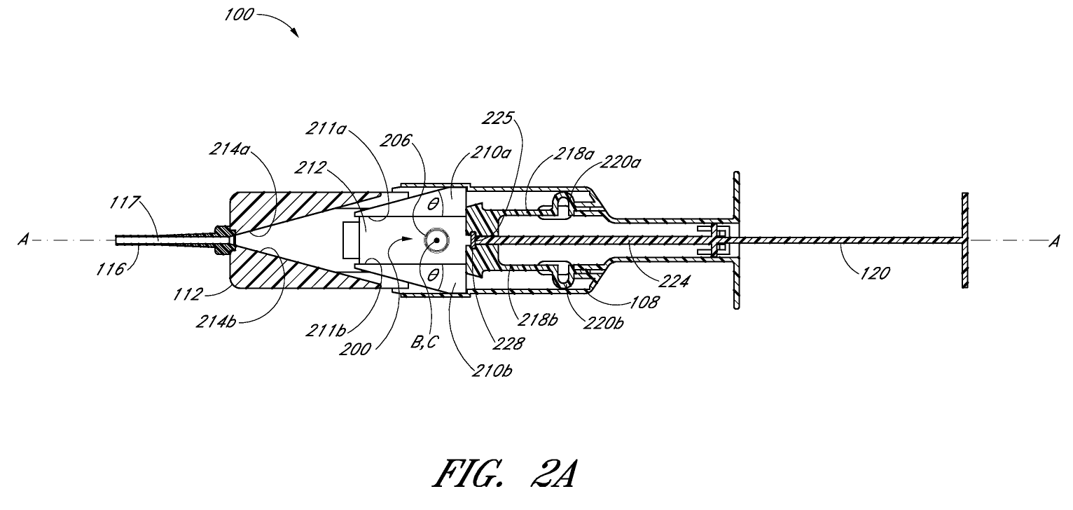

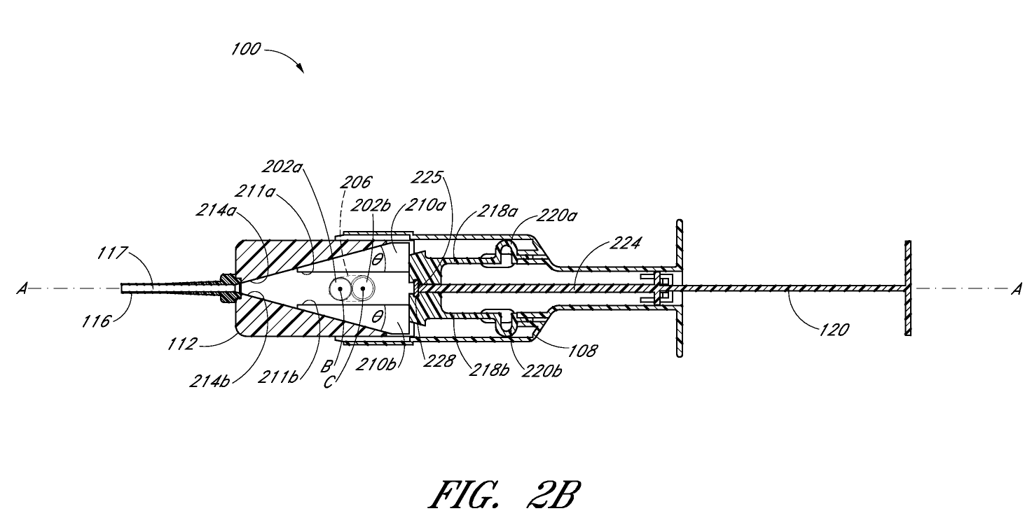

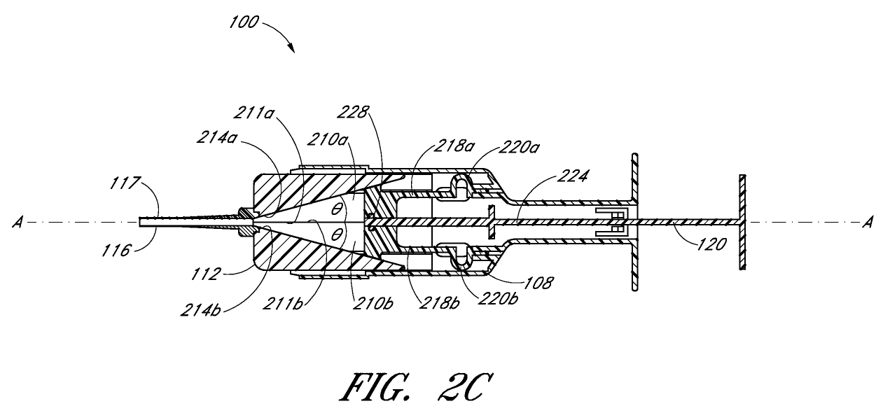

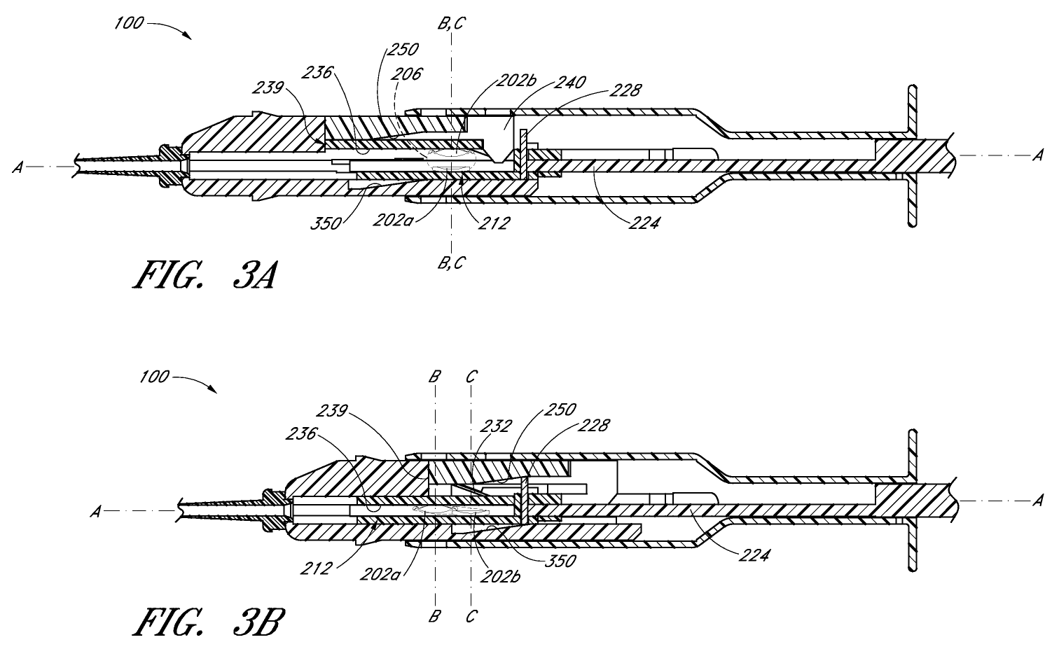

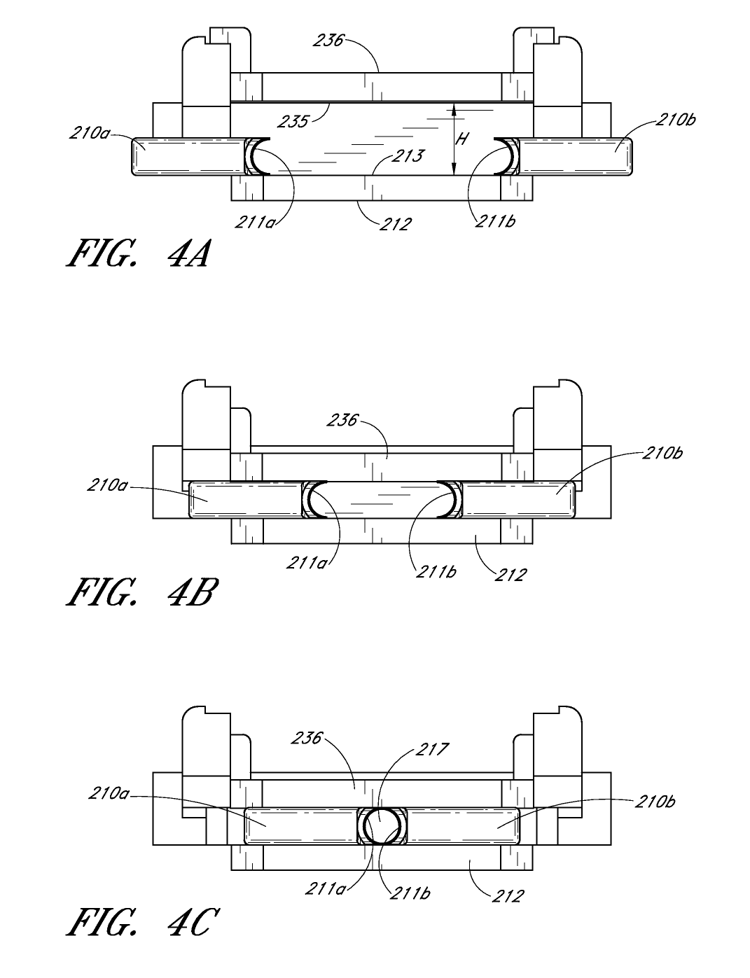

FIGS. 2A-2C are top cross-sectional views, FIGS. 3A-3B are side cross-sectional views, and FIGS. 4A-4C are front views (along the longitudinal axis A-A) of the embodiment of the injector 100 shown in FIGS. 1A-1C. FIGS. 2A-4C schematically illustrate stages of the compaction of an IOL 200 in the injector 100. In the illustrated embodiment, the IOL 200 is an example of an accommodating intraocular lens comprising a first viewing element 202a and a second viewing element 202b, which are interconnected by biasing members 206 (see, e.g., FIGS. 2B and 3A which depict the biasing members 206 with dashed lines). One or both of the viewing elements 202a, 202b may comprise an optic having refractive power. The first viewing element 202a has a first viewing axis B-B, and the second viewing element 202b has a second viewing axis C-C. The first and the second viewing axes B-B, C-C are each generally orthogonal to and centered on the respective first and second viewing elements 202a, 202b. When the IOL 200 is in an unstressed condition (see, e.g., FIG. 2A), the first and the second viewing axes B-B and C-C are generally aligned (e.g., the axes B-B and C-C are substantially coaxial or colinear). Because FIGS. 2A-2C are top views showing a plane generally orthogonal to the first and the second viewing axes B-B and C-C, the viewing axes B-B, C-C are each depicted as a point labeled B or C, respectively.

An IOL of this type may be implanted in the ciliary capsule such that the biasing members 206 maintain one of the viewing elements 202a, 202b against the anterior region of the ciliary capsule and the other of the viewing elements 202a, 202b against the posterior region of the ciliary capsule. The biasing members 206 may be configured to be spring-like to allow the separation between the viewing elements 202a, 202b to change in response to changes in the shape of the ciliary capsule that occur during accommodation. In some embodiments, the IOL may comprise a frame to hold and/or separate the viewing elements 202a, 202b. The frame may be in addition to or instead of the biasing members 206.

Embodiments of the injector 100 may be used to compact and inject IOLs that are different than the example IOL 200 depicted in FIGS. 2A-4C. For example, the IOL 200 may comprise a single-lens, dual-lens, or multiple-lens IOL of the accommodating or non-accommodating type. The IOL 200 may have two or more interconnected viewing elements or two or more interconnected optics. One, both or all of the viewing elements of the IOL 200 may comprise an optic or lens having a power, e.g., a refractive power and/or a diffractive power. Also, one, both or all of the viewing elements may comprise an optic with a surrounding or partially surrounding perimeter frame member or members, with some or all of the interconnecting members attached to the frame member(s). As a further alternative, one of the viewing elements may comprise a perimeter frame with an open/empty central portion or void located on the optical axis, or a perimeter frame member or members with a zero-power lens or transparent member therein. In still further variations, one of the viewing elements may comprise only a zero-power lens or transparent member. Many variations of IOLs designs and configurations may be used with embodiments of the injector 100 described herein.

In some embodiments, the IOL 200 may comprise any of the various embodiments of accommodating intraocular lenses described in U.S. Pat. No. 7,198,640, issued Apr. 3, 2007, entitled “ACCOMMODATING INTRAOCULAR LENS SYSTEM WITH SEPARATION MEMBER,” or any of the various embodiments of accommodating intraocular lenses described in U.S. Patent Application Publication No. US 2005/0234547, published Oct. 20, 2005, entitled “INTRAOCULAR LENS.” The entire disclosure of the above-mentioned patent and the entire disclosure of the above-mentioned patent application publication are hereby incorporated by reference herein and made a part of this specification. In still other embodiments, the IOL 200 may comprise a single-optic system, of the accommodating or non-accommodating type.

Compaction and delivery of the IOL 200 in the embodiment of the injector 100 shown in FIGS. 1A-4C will now be briefly summarized. When the injector 100 is in the open position (see, e.g., FIGS. 1A, 2A, 3A, and 4A), the IOL 200 can be held in an unstressed condition in the first housing 108. When the IOL 200 is in the unstressed condition, the first and the second viewing axes B-B, C-C can be generally orthogonal to the longitudinal axis A-A of the injector 100 (see, e.g., FIGS. 2A and 3A). Before compacting the IOL 200, a medical practitioner optionally may apply a hydrating solution through the port 128 to activate lubricious substances (if present) that may be coated on moving parts of the injector 100. In some embodiments, the lubricous substance may be located on a fixed structure, such as a surface of the lumen 117. In some embodiments, the hydrating solution itself acts as a lubricant.

The medical practitioner then relatively moves the first and the second housing 108, 112 along the longitudinal axis A-A so that the injector 100 moves from the open position (see, e.g., FIGS. 1A, 2A, 3A, and 4A) to the closed position (see, e.g., FIGS. 1B, 2C, and 4C). As the injector 100 moves from the open to the closed position, the viewing elements 202a, 202b of the IOL 200 are first displaced relative to each other along the longitudinal axis A-A (see, e.g., FIGS. 2B, 3B, and 4B), and then the (thus displaced) IOL 200 is compacted while it remains substantially along the longitudinal axis A-A (see, e.g., FIGS. 2C and 4C).

During the compaction process, some embodiments of the injector 100 utilize a plunger lock that prevents the plunger 120 from inadvertently being depressed before compaction is complete. When the injector 100 is in the closed position (see, e.g., FIGS. 1B, 2C, and 4C), compaction of the IOL 200 is complete, and the plunger lock (if used) unlocks. The medical practitioner may then depress the plunger 120 to force the displaced and compacted IOL 200 along the longitudinal axis A-A and through the injection nozzle 116 to a desired location in the eye (see, e.g., the delivered position of the injector 100 shown in FIG. 1C).

FIGS. 2A, 3A, and 4A are top, side, and front views schematically illustrating the embodiment of the injector 100 shown in FIGS. 1A-1C, in the open position. FIGS. 2B, 3B, and 4B are top, side, and front views schematically illustrating the injector 100 in a “displaced” position in which the viewing elements 202a, 202b of the IOL 202 are longitudinally displaced relative to each other. FIGS. 2C and 4C are top and front views of the injector 100 in the closed position. The top and side sectional views are taken along central planes of the injector 100, and the front views are taken from the front of the injector 100 along the longitudinal axis A-A. As shown in these figures, the first housing 108 of the injector 100 comprises a substantially planar support member 212 for supporting the IOL 200. In this embodiment, the first viewing element 202a of the IOL 200 is supported by the support member 212. When the injector 100 is in the open position, the IOL 200 is in a substantially unstressed condition, and the second viewing element 202b is disposed over the first viewing element 202a. The first and the second viewing axes B-B, C-C are generally aligned with each other and are each generally orthogonal to the longitudinal axis A-A. In some embodiments, the first viewing element 202a comprises a posterior optic and the second viewing element 202b comprises an anterior optic. The terms “anterior” and “posterior” are derived from the positions preferably assumed by the viewing elements 202a, 202b upon implantation of the IOL 200 in an eye.

The injector 100 comprises a lens engagement member 236 that is configured to engage the second viewing element 202b, but not the first viewing element 202a, as the injector 100 is moved from the open position (see, e.g., FIGS. 2A, 3A, and 4A) to the “displaced” position (see, e.g., FIGS. 2B, 3B, and 4B). In the displaced position, the lens engagement member 236 has moved both longitudinally along the axis A-A and vertically downward so as to longitudinally displace the second viewing element 202b relative to the first viewing element 202a. In the displaced position, the first and the second viewing axes B-B and C-C are longitudinally displaced relative to each other but remain substantially parallel. In some embodiments, it is advantageous if the anterior optic (e.g., the viewing element 202b) is displaced rearward of the posterior optic (e.g., the viewing element 202a) so that the posterior optic is injected before the anterior optic. In some embodiments, the first and the second viewing elements 202a, 202b are relatively displaced so that the viewing elements 202a, 202b do not “overlap,” as viewed along the viewing axis B-B, C-C of either element. In certain embodiments, the viewing elements 202a, 202b are relatively displaced so that the viewing elements 202a, 202b are in a substantially planar, “side-by-side” arrangement (either overlapping or non-overlapping) such that the vertical thickness of the IOL 200 is reduced or minimized (see, e.g., FIG. 3B).

The first housing 108 also comprises a first compaction member 210a and a second compaction member 210b that are relatively movable with respect to each other. In the illustrated embodiment, the first and the second compaction members 210a, 210b are generally wedge-shaped and form a wedge angle θ at a distal end of the wedge (see, e.g., FIG. 2A). The wedge angle θ is about 15 degrees in some embodiments. In certain embodiments, the wedge angle θ is in a range from about 10 degrees to about 20 degrees. In other embodiments, the wedge angle θ may be in a range from about 5 degrees to about 10 degrees, from about 10 degrees to about 15 degrees, from about 15 degrees to about 20 degrees, from about 20 degrees to about 25 degrees, or some other range. In the illustrated embodiment, the first and the second compaction members 210a, 210b have substantially equal wedge angles θ. In other embodiments, the compaction members 210a, 210b may each have different wedge angles. In yet other embodiments, one or both of the compaction members 210a, 210b may be shaped differently from a wedge.

The second housing 112 has surfaces 214a and 214b that are angled at the wedge angle θ so as to cooperatively engage the compaction members 210a and 210b, respectively, as the injector 100 is moved from the open position to the closed position (see, e.g., FIGS. 2A-2C, and 4A-4C). Relative longitudinal movement of the first and the second housings 108, 112 causes the surfaces 214a, 214b to force the compaction members 210a, 210b toward each other along a direction transverse to the longitudinal axis A-A (see, e.g., FIGS. 4A-4C). The first and the second compaction members 210a, 210b have first and second compaction surfaces 211a, 211b, respectively, that are substantially parallel to the longitudinal axis A-A. When the injector 100 is in the open position, the first and the second compaction surfaces 211a, 211b are spaced from each other by a first distance (transverse to the longitudinal axis A-A), and when the injector 100 is in the closed position, the first and the second compaction surfaces 211a, 211b are spaced from each other by a second distance (transverse to the longitudinal axis A-A), which is less than the first distance. In some embodiments, the first distance is greater than a diameter of the IOL 200 (see, e.g., FIG. 2A) so that the compaction surfaces 211a, 211b do not engage the IOL 200 when the injector 100 is in the open position. Such embodiments advantageously permit the IOL 200 to remain in the substantially unstressed condition while being stored. In certain embodiments, the first and the second compaction surfaces 211a, 211b are spaced from a projection of the injection lumen 117 when the injector 100 is in the open position and are spaced substantially along the projection of the injection lumen 117 when the injector 100 is in the closed position.

As the first and the second compaction members 210a, 210b are transversely forced toward each other by the angled surfaces 214a, 214b, the IOL 200 is substantially trapped between the first and the second compaction surfaces 211a, 211b (in the transverse direction) and between the lens engagement member 236 and the support member 212 (in the vertical direction). Consequently, convergence of the first and the second compaction members 210a, 210b causes the (displaced) IOL 200 to be compacted between the first and the second compaction surfaces 211a, 211b. In the closed position of the injector 100, the second (transverse) distance between the compaction members 210a, 210b may be selected so that the IOL 200 is sufficiently compacted to permit delivery through the injection lumen 117 of the injection nozzle 116. In some embodiments of the injector 100, the compaction members 210a, 210b exert a compaction force on the IOL 200 that may be in a range from about 1 pound to about 2 pounds. Although both the first and the second compaction members 210a, 210b are configured to be movable in the illustrated injector embodiment, in other embodiments, one of the compaction members is movable and the other compaction member is fixed.

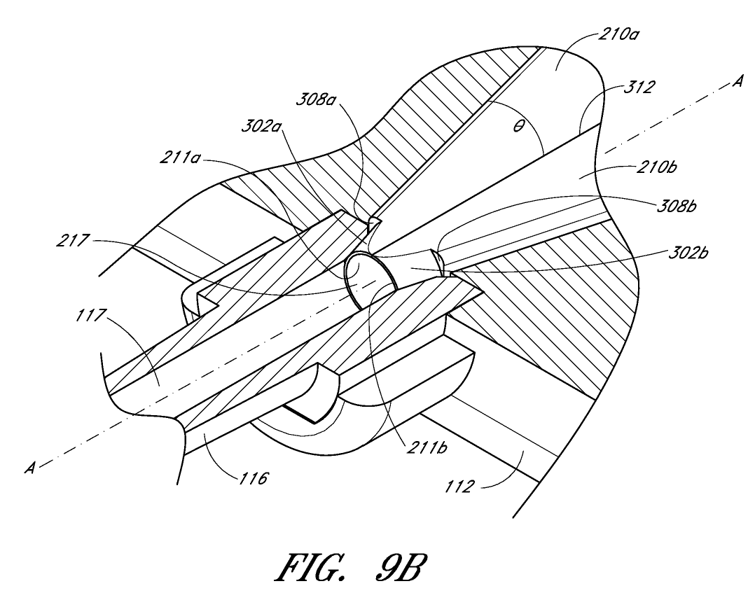

In certain embodiments, the first and the second compaction surfaces 211a, 211b are in the form of a half-channel (e.g., C-shaped as shown in FIGS. 4A-4C). When the injector 100 is in the open position, the first and the second compaction surfaces 211a, 211b are spaced apart from a projection of the injection lumen 117 of the injection nozzle 116 (see, e.g., FIG. 4A). As the injector 100 is moved to the closed position, the first and the second compaction members 210a, 210b converge, and the first and the second compaction surfaces 211a, 211b form a delivery channel 217 along the longitudinal axis A-A of the injector 100 (see, e.g., FIG. 4C). The delivery channel 217 therefore is substantially aligned with the projection of the injection lumen 117 of the injection nozzle 116 and with the plunger 120. The delivery channel 217 advantageously may have a cross-section that substantially matches the internal cross-section of the injection lumen 117 of the injection nozzle 116 (see, e.g., FIG. 9B).

When the injector 100 is in the closed position, the delivery channel 217 holds the displaced, compacted IOL 200, which is ready for further distal longitudinal movement by distal movement of the plunger 120. The plunger 120 may comprise a plunger rod 224 having a cross-section that substantially matches the cross-section of the delivery channel and the injection lumen 117. Depression of the plunger 120 drives a tip 225 of the plunger rod 224 forward into the delivery channel 217 between the compaction surfaces 211a, 211b and against the displaced, compacted IOL 200. Further depression of the plunger 120 urges the displaced, compacted IOL 200 through the delivery channel 217 and into the injection lumen 117 of the injection nozzle 116. The end of the injection nozzle 116 may be inserted into an eye of the patient for delivery of the IOL 200 from the tip of the nozzle 116. Certain embodiments of the injector 100 comprise a plunger lock 228, which prevents distally-directed movement of the tip 225 of the plunger rod 224 until the injector 100 is in the closed position and the IOL 200 is fully compacted. Further details of an embodiment of the plunger lock are described with reference to FIGS. 10A and 10B.

When the injector 100 is in the closed position, the natural resiliency of the material forming the IOL 200 causes the compacted IOL 200 to exert an outwardly directed force tending to push apart the compaction members 210a, 210b. This outwardly directed force may be sufficiently large in some cases to cause the compaction members 210a, 210b to separate and move slightly rearward along the longitudinal axis A-A. In such cases, the IOL 200 will become at least partially uncompacted and portions of the IOL 200 may be forced between edges of the compaction surfaces 211a, 211b, which may lead to cutting and/or tearing of the IOL 200. Accordingly, to avoid such possible disadvantages, certain embodiments of the injector 100 comprise retention members 218a and 218b that are configured to apply a distally-directed, retention force on the lens compaction members 210a, 210b when the injector 100 is in the closed position (see, e.g., FIGS. 2A-2C). The retention force may be selected to be sufficiently large to retain the compaction members 210a, 210b in the compacted arrangement (shown in, e.g., FIG. 2C) and to prevent the IOL 200 from at least partially uncompacting.

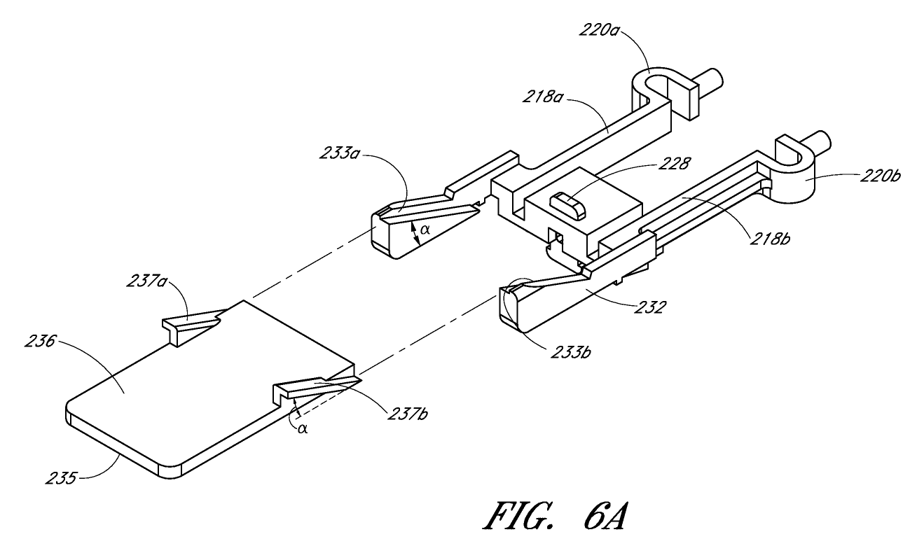

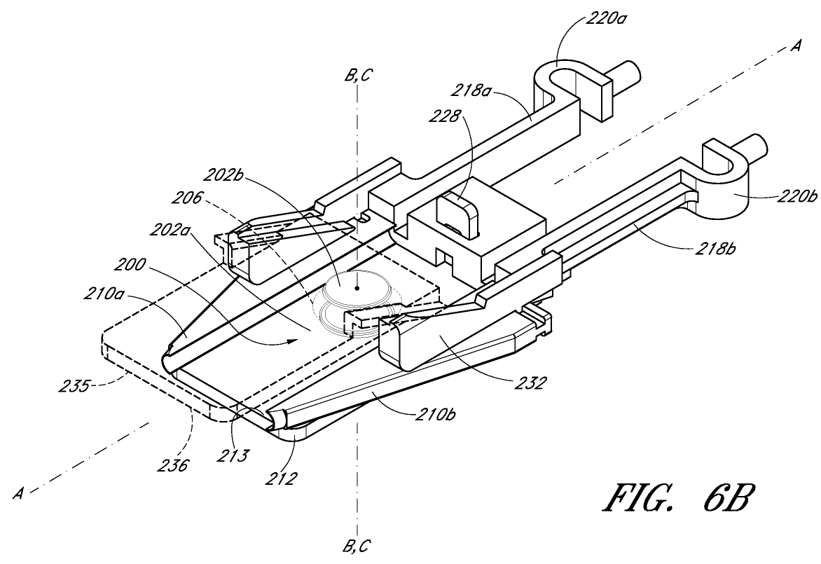

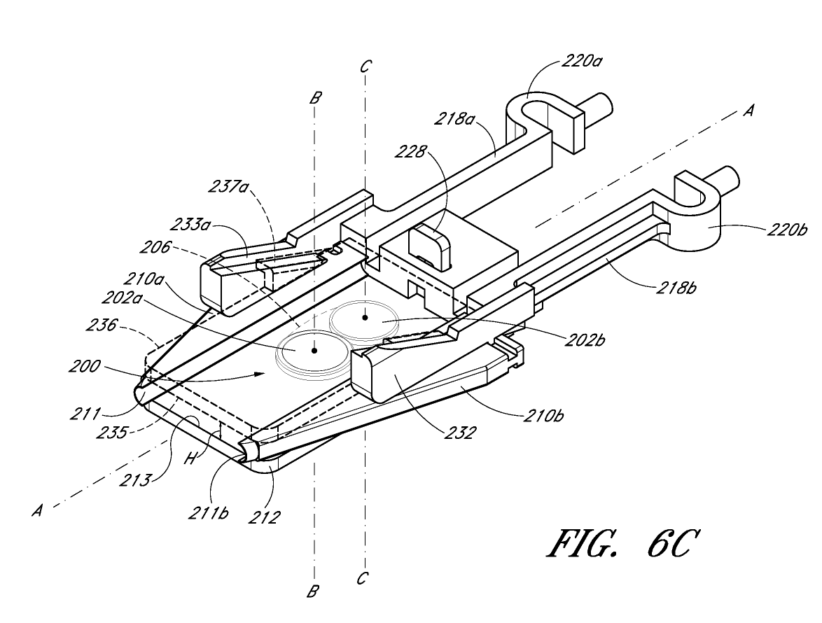

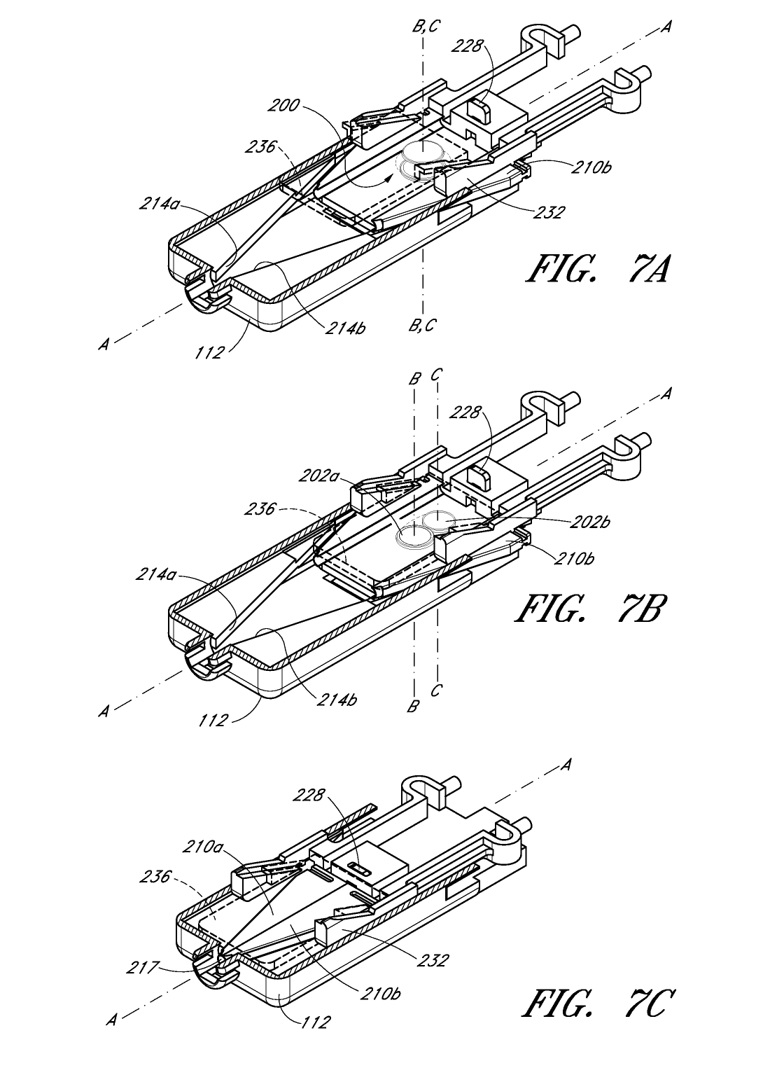

In the embodiment shown in FIGS. 2A-2C, the retention members 218a, 218b are elongated elements located in the first housing 108. Each retention member 218a, 218b has a distal end that contacts one or both of the compaction members 210a, 210b, either directly or through an intermediary structure (see, e.g., the ramp 232 in FIGS. 6A-7C). Each retention member 218a, 218b has a proximal end fixed at a rear surface of the first housing 108. Each of the retention members 218a, 218b has a U-shaped portion that is configured to flex slightly so as to provide a spring-like force in the distal, longitudinal direction. When the injector 100 is in the open position, the retention members 218a, 218b are not under compression (or tension), and the retention members 218a, 218b do not apply a retention force on the compaction members 210a, 210b. When the injector 100 is moved to the closed position, the retention members 218a, 218b are placed under a slight compression, causing the U-shaped portions to flex slightly, thereby causing the retention members 218a, 218b to exert a distally-directed, longitudinal retention force on the compaction members 210a, 210b.

A desired amount of retention force may be provided by suitably selecting the structural properties of the retention members 218a, 218b and/or their U-shaped portions. In other embodiments, one, three, four or more retention members may be used. Also, the retention member may be formed differently than shown in the example embodiment of FIGS. 2A-2C. For example, the retention members 218A, 218B may be formed from a resilient material (e.g., an elastomer) and the U-shaped portion may not be used in some embodiments. In other embodiments, the retention member(s) may comprise one or more springs configured to exert the retention force when the injector 100 is in the closed position. In still other embodiments, one (or both) of the housings 108, 112 may comprise features (e.g., detents) that engage the retention members 218a, 218b to maintain the members in the compacted arrangement when the injector 100 is in the closed position.

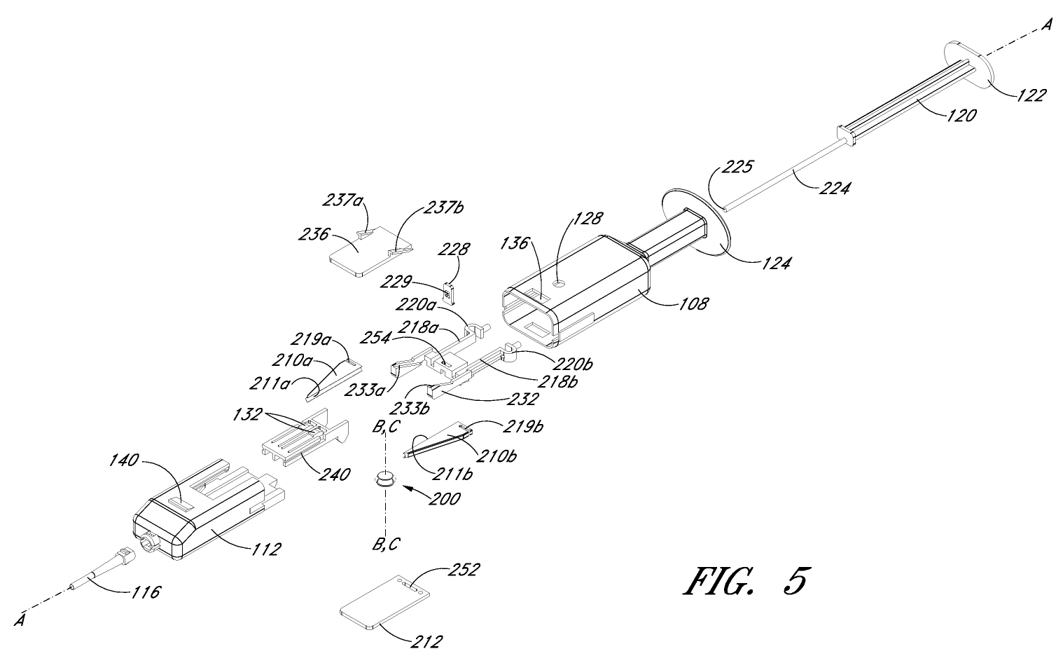

FIG. 5 is an exploded view that schematically illustrates an embodiment of an injector 100 that may be generally similar to the injector embodiment of FIGS. 1A-4C. FIGS. 6A-10B further illustrate various aspects of embodiments of components of the injector 100 shown in FIG. 5.

FIGS. 6A-6C are perspective views that schematically illustrate how the lens engagement member 236 is configured to move longitudinally and vertically to displace the second viewing element 212b relative to the first viewing element 212a. As shown in FIG. 6A, the lens engagement member 236 comprises angled flanges 237a and 237b that engage and slide downward on ledges 233a and 233b of a ramp 232. In the illustrated embodiment, the angled flanges 237a, 237b and the ledges 233a, 233b are formed at an angle α. The angle α is about 20 degrees in certain embodiments. In some embodiments, the angle α may be in a range from about 1 degree to about 45 degrees, from about 10 degrees to about 30 degrees, from about 15 degrees to about 25 degrees, or in some other range. FIGS. 6B and 6C show in more detail how the lens engagement member 236 and the ramp 232 cooperate to longitudinally displace the second viewing element 202b. FIG. 6B schematically illustrates these components when the injector 100 is in the open position, and FIG. 6C schematically illustrates these components when the injector 100 is in the displaced position (having moved partway toward the closed position). The lens engagement member 236 is shown with dashed lines in FIGS. 6B and 6C to more readily show components lying below the member 236.

As can be seen in FIG. 6B, the IOL 200 is disposed so that the first viewing element 202a rests on an upper surface 213 of the support member 212, with the second viewing element 202b vertically above the first viewing element 202a. When the injector 100 is in the open position, the lens engagement member 236 is disposed in the second housing 112 in a carrier 240 (shown in FIGS. 8A-8C). A lower surface 235 of the lens engagement member 236 is vertically spaced (by a vertical distance H depicted in FIGS. 4A and 6C) from the upper surface 213 of the support member 212 to permit the IOL 200 to be disposed therebetween. In some embodiments, the lower surface 235 lightly touches the second viewing element 202b when the injector 100 is in the open position, but with insufficient force to substantially disturb the IOL 200 from the unstressed condition. In some embodiments, the lower surface 235 does not touch or compress the second viewing element 202b when the injector 100 is in the open position, which advantageously reduces the likelihood of compression set of the IOL 200 during storage. In other embodiments, the lower surface 235 may compress the second viewing element 202b.

A ridge-like feature 239 (shown in FIGS. 3A AND 3B) is formed in the second housing 112 and engages the distal edge of the lens engagement member 236. As the injector is moved from the open position (see, e.g., FIG. 6B) to the displaced position (see, e.g., FIG. 6C), the feature 239 urges the lens engagement member 236 longitudinally rearward (or maintains the position of the feature 239 while the housing 108 moves forward), and the lens engagement member 236 slides vertically down the ramp 232, thereby decreasing the vertical distance H relative to its initial value when the injector 100 is in the open position. The lower surface 235 of the lens engagement member 236 engages the second viewing element 202b, but not the first viewing element 202a. As the injector 100 is moved to the displaced position, the rearward and downward movement of the lower surface 235 urges the second viewing element 202b rearward relative to the first viewing element 202a, thereby displacing the second viewing axis C-C relative to the first viewing axis B-B. As the lens displacement member 236 slides downward on the ramp 232, the distal edge of the lens displacement member 236 disengages from the feature 239 in the second housing 112 when the injector 100 has displaced the IOL 200. Accordingly, further relative longitudinal movement of the housings 108, 112 does not further longitudinally displace the lens engagement member 236 relative to the IOL 200, the lens compaction members 210a, 210b, or the support member 212. In some embodiments, the viewing elements 202a, 202b are fully displaced relative to each other when the injector 100 is in the displaced position (see, e.g., FIG. 6C). In other embodiments, the viewing elements 202a, 202b are not fully displaced, e.g., there may be at least partial overlap between the first viewing element 202a and the second viewing element 202b when the injector 100 is in the displaced position.

In various embodiments of the injector 100, the angle α of the angled flanges 237a, 237b and the ledges 233a, 233b and the initial vertical height H between the lower surface 235 and the upper surface 213 may be selected to achieve various design objectives. For example, as the angle α becomes shallower (for a given initial value of the vertical distance H), the overall length of the injector 100 tends to increase to accommodate movement of the lens engagement member 236 down the more shallow ramp provided by the angled flanges 237a, 237b and the ledges 233a, 233b. As the angle α becomes larger (for a given initial value of the vertical distance H), the relative displacement between the viewing elements 202a, 202b (when in the displaced position) tends to decrease, because the lens engagement member 236 tends to have less longitudinal movement (along the axis A-A) as it moves down the steeper ramp provided by the angled flanges 237a, 237b and the ledges 233a, 233b. In certain embodiments, values of the angle α and the initial vertical distance H may be selected so that the IOL 200 is substantially uncompressed when the injector 100 is in the home position, and so that the viewing elements 202a, 202b are substantially fully displaced when the injector 100 is in the displaced position. For example, in certain such embodiments, the angle α is in a range from about 18 degrees to about 22 degrees (e.g., about 20 degrees in one case), and the initial value of the vertical distance H is in a range from about 0.12 inches to about 0.16 inches (e.g., about 0.14 inches in one case).

In the fully displaced position shown in FIG. 6C, the lower surface 235 of the lens engagement member 236 rests on upper surfaces of the lens compaction members 210a and 210b. The displaced IOL 200 is “trapped” between the upper surface 213 of the support member 212 and the lower surface 235 of the lens engagement member 236 as the lens compaction members 210a, 210b converge on the IOL 200 (as the injector 100 moves from the displaced position to the closed position).

FIGS. 7A and 7B are perspective views that further schematically illustrate displacement of the viewing elements 202a, 202b of the IOL 200 by the lens engagement member 236 as the injector 100 moves from the open position (see, e.g., FIG. 7A) to the displaced position (see, e.g., FIG. 7B). FIG. 7C is a perspective view that schematically illustrates compaction of the displaced IOL 200 by the lens compaction members 210a, 210b as the injector 100 moves from the displaced position (see, e.g., FIG. 7B) to the closed position (see, e.g., FIG. 7C). As the injector 100 moves to the closed position, the angled surfaces 214a, 214b of the second housing 112 engage the outer angled edges of the lens compaction members 210a, 210b, which causes the compaction members 210a, 210b to be forced toward the longitudinal axis A-A. The displaced IOL 200 is compacted in the delivery channel 217 formed between the lens compaction surfaces 211a, 211b (see, e.g., FIGS. 4C and 7C).

FIG. 8A is a perspective view of an embodiment of a mechanism for displacing and compacting the IOL 200 within the injector 100. FIG. 8B is an exploded view of the mechanism illustrated in FIG. 8A (see also, e.g., the exploded view in FIG. 5). The lens displacement member 236 fits under the carrier 240, which attaches to a slot in an upper surface of the second housing 112 (see, e.g., FIG. 5). As described above with reference to FIG. 1A, the carrier 240 includes the engagement element 132 used to lock or temporarily retain the injector 100 in the open position. In this embodiment, the engagement element 132 comprises ramped protrusions 132a formed on proximal ends of tabs in the upper surface of the carrier 240. The protrusions 132a can be slightly depressed to permit them to slide under the upper surface of the first housing 108, thereby permitting the injector 100 to be moved from the open position (see, e.g., FIGS. 1A and 1B). The injector 100 also can be urged from the open position toward the closed position by applying a compressive force to the injector 100 along the longitudinal axis A-A. In response to the compressive force, the ramps 132a on the rearward ends of the engagement element 132 move below the upper portion of the first housing 108, thereby permitting the first and the second housings 108, 112 to move relative to each other.

As illustrated in FIGS. 8A and 8B, the first and second lens compaction members 210a, 210b are disposed between the support member 212 and the ramp 232. To prevent the compaction members 210a, 210b from moving relative to the ramp 232 during displacement/compaction of the IOL 200, proximal ends of the compaction members 210a, 210b comprise transverse slots 219a, 219b, respectively, that engage respective tabs 221a, 221b formed on the ramp 232. The transverse slots 219a, 219b and the tabs 221a, 221b constrain the lens compaction members 210a, 210 to move transversely, rather than longitudinally, relative to the ramp 232. Thus, the ramp 232 can transmit the retention force to the compaction members 210a, 210b.

In the embodiment shown in FIGS. 8A and 8B, the retention members 218a, 218b are formed as rearwardly extending, elongated portions of the ramp 232. As described above, when the injector 100 is in the closed position, the retention members 218a, 218b are placed under compression and exert a forward-directed retention force. In this embodiment, the retention force is transmitted to the ramp 232 and thereby to the lens compaction members 210a, 210b, because the members 210a, 210b are coupled to the ramp 232 by the transverse slots 219a, 219b and corresponding tabs 221a, 221b.

FIG. 8C is a bottom perspective view of an embodiment of the carrier 240 that may be used with the automatic plunger lock mechanism described with reference to FIGS. 10A-10D. In this embodiment, the carrier 240 comprises a substantially central rib 242 having an inclined surface 250. As will be further described below, the inclined surface 250 is configured to engage an upper end of the plunger lock 228 as the injector 100 is moved from the open position to the closed position.

FIGS. 9A and 9B are closeup cutaway views schematically illustrating convergence and engagement of distal ends of the lens compaction members 210a, 210b with the injection lumen 117 of the injection nozzle 116 in an embodiment of the injector 100. FIG. 9A is a closeup view just prior to the closeup view in FIG. 9B at the time the injector 100 is in the closed position. In this embodiment, the distal ends of the compaction members 210a, 210b each comprise an angled surface 302a, 302b, respectively, that is configured to engage an angled mating surface 306 adjacent a proximal end of the injection lumen 117. In certain embodiments, the angled surfaces 302a, 302b are shaped as portions of a truncated cone that engages the mating surface 306, which may be conically-shaped to receive the surfaces 302a, 302b. The angled surfaces 302a, 302b advantageously may be formed at substantially the same angle with respect to the longitudinal axis A-A as the angled mating surface 306. For example, in certain embodiments, the surfaces 302a, 302b, and 306 are formed substantially at the wedge angle θ, which advantageously provides a smooth, “feathered” transition between the distal ends of the lens compaction members 210a, 210b and the injection lumen 117.

In certain embodiments, the distal ends of the lens compaction members 210a, 210b also comprise respective angled ledges 308a, 308b that are configured to mate with an angled transition surface 310 formed rearward of the mating surface 306 (see, e.g., FIG. 9A). The angled ledges 308a, 308b and the angled transition surface 310 may each be formed at the same angle with respect to the longitudinal axis A-A. In some embodiments, this angle is greater than the wedge angle θ but less than about 90 degrees (see, e.g., FIG. 9A). Embodiments in which this angle is less than 90 degrees advantageously reduce the likelihood that an edge can catch or cut the IOL 200 as it passes from the delivery lumen 217 to the injection lumen 117. In certain embodiments, the transition surface 310 may be shaped differently than shown in FIGS. 9A, 9B. For example, the transition surface 310 may comprise curved or arcuate portions that mate with the ledges 308a, 308b.

In the illustrated embodiment, when the injector 100 is in the closed position (FIG. 9B), the lens compaction members 210a, 210b meet along seams 312 that are substantially parallel to (but displaced from) the longitudinal axis A-A (a lower seam is present but not shown in FIG. 9B). The delivery channel 217 is formed between the lens compaction surfaces 211a, 211b of the lens compaction members 210a, 210b. In certain embodiments, the internal cross-section of the delivery channel 217 substantially matches the internal cross-section of the injection lumen 117, thereby forming a substantially smooth and uniform delivery path which may prevent tearing, cutting, or otherwise damaging the compacted IOL 200 as it passes from the delivery channel 217 to the injection lumen 117. In certain embodiments, the internal cross-sections of the injection lumen 117 and the delivery channel 217 are substantially circular. In certain such embodiments, the circular cross-sections may have diameters in a range from about 0.05 inches to about 0.10 inches.

In the closed position of the injector 100, the angled surfaces 302a, 302b of the lens compaction members 210a, 210b meet to form a truncated cone that engages the angled mating surface 306 of the injection lumen 117 (see, e.g., FIGS. 9A and 9B). By forming the angled mating surface 306 at substantially the same angle as the angled surfaces 302a, 302b, the angled mating surface 306 will more intimately engage the surfaces 302a, 302b and will better support the angled surfaces 302a, 302b from distortion and/or deflection when the compacted IOL 200 passes through this region. Additionally, the retention force applied by the retention members 218a, 218b will tend to urge the distal ends of the lens compaction members 210a, 210b into the injection lumen 117, which further provides a tight fit. Such embodiments place the lens engagement members 210a, 210b under transverse compression, which tends to urge the members 210a, 210b securely together along the seams 312 (see, e.g., FIG. 9B) and reduces the likelihood that portions of the compressed IOL 200 will escape out of seams 312 of the delivery channel 217, which could damage the IOL 200.

As can be seen in FIG. 4C, the lens compaction members 210a, 210b are disposed between a lower surface 235 of the lens engagement member 236 and an upper surface of the support member 212, when the injector 100 is in the closed position. The lens engagement member 236 and the support member 212 therefore tend to prevent vertical buckling by one or both of the lens engagement members 210a, 210b when the compacted IOL 200 is pushed through the delivery channel 217 and into the injection lumen 117. By reducing the likelihood of such buckling, possible damage to the IOL 200 can be reduced.

As described above, certain embodiments of the injector 100 comprise a plunger lock that prevents inadvertent depression of the plunger 120 before the IOL 200 is fully compacted in the delivery channel 217. Accordingly, a plunger lock advantageously may reduce possible damage to the IOL 200, for example, when the injector 100 is in the open position and the IOL 200 is being stored for future use. In some embodiments, the plunger lock comprises a user-removable clip that attaches to the plunger 120 and prevents depression or advancement of the plunger 120 while the clip is in place. A possible disadvantage of such embodiments is that the clip must be manually removed by the medical practitioner during the procedure to deliver the IOL to the patient's eye.

FIGS. 10A and 10B are cross-section views that schematically illustrate an embodiment of an automatic plunger lock mechanism that requires no user intervention apart from moving the injector 100 from the open position to the closed position. FIGS. 10C and 10D are close-up views of the plunger lock mechanism illustrated in FIGS. 10A and 10B, respectively. The plunger 120 is locked in FIGS. 10A, 10C and unlocked in FIGS. 10B, 10D. In this embodiment, the locked positions of the injector 100 correspond to the open position (see, e.g., FIG. 2A) and the displaced position (see, e.g., FIG. 2B). The unlocked position of the injector 100 corresponds to the closed position in which the IOL 200 is fully compacted. In the locked position, the plunger 120 is prevented from being advanced, and in the unlocked position, the plunger 120 is permitted to advance distally along the longitudinal axis A-A. Accordingly, in this embodiment, the IOL 200 is prevented from being injected to the surgical site until the IOL 200 is fully compacted and the lens compaction members 210a, 210b have converged to form the delivery channel 217 for the compacted IOL 200.

The embodiment of the plunger lock mechanism schematically illustrated in FIGS. 10A-10D comprises the plunger lock 228, which is a solid structure having an opening 229 sized and shaped to permit passage of at least a distal portion of the plunger rod 224. The plunger lock 228 is configured to move vertically through slot 252 in the support member 212 and slot 254 in the ramp 232 (see, e.g., the exploded view in FIG. 8B). The bottom of the plunger lock 228 rests on an inclined surface 350 formed in the bottom of the second housing 112 (see, e.g., FIGS. 10A, 10B and 3A, 3B). The top of the plunger lock 228 is configured to engage the inclined surface 250 of the central rib 242 of the carrier 240 (shown in FIG. 8C). When the injector 100 is in the open position, the plunger lock 228 is located at the top of the inclined surface 350. The plunger lock 228 extends through the slots 252 and 254 so that the opening 229 is positioned above the tip 225 of the plunger rod 224 (see, e.g., FIGS. 10A, 10C). If the plunger 120 were depressed, the tip 225 of the plunger rod 224 would contact a solid portion of the plunger lock 228 and be prevented from further distal longitudinal movement. As the injector 100 is moved toward the closed position, the first and second housings 108, 112 move together, and the top of the plunger lock 228 engages the inclined surface 250, which urges the plunger lock 228 to slide down the inclined surface 350 (see, e.g., FIG. 10B). In some embodiments, the inclined surfaces 250 and 350 may be formed at the same angle of inclination so that the surfaces 250 and 350 mutually cooperate to smoothly control the vertical movement of the plunger lock 228. In certain embodiments, some or all of the inclined surfaces 250, 350, the plunger lock 228, and the slots 252, 254 may be coated with a lubricious substance to reduce friction as the automatic plunger lock operates.

The downward vertical movement of the plunger lock 228 lowers the opening 229 until the opening 229 is at the same (vertical) level as the tip 225 of the plunger rod 224 (see, e.g., FIG. 10D). When the plunger lock 228 is positioned as shown in FIG. 10B (e.g., the closed position of the injector 100), the tip 225 of the plunger rod 224 can pass through the opening 229 as schematically illustrated in FIG. 10D. Distal longitudinal movement of the plunger 120 can occur at this point, and the plunger 120 becomes unlocked. In this embodiment, the plunger lock mechanism automatically unlocks the plunger 120, with no additional user input required, apart from the movement of the injector 100 from the open position to the closed position. The plunger 120 can be depressed by the medical practitioner to advance the compacted IOL 200 through the delivery chamber 217 and the injection lumen 117.

FIG. 11 is a flowchart that schematically illustrates an embodiment of a method 1100 for preparing an IOL for implantation into an eye. The method 1100 may be used with any of the embodiments of the injector 100 and/or any of the embodiments of an IOL described herein. The IOL may be disposed (or stored) along a longitudinal axis of the injector 100. If the IOL comprises two or more interconnected viewing elements having respective viewing axes, the viewing axes may be substantially colinear and may be substantially orthogonal to the longitudinal axis of the injector 100. In optional block 1110, a hydrating solution may be applied to the injector 100 to lubricate movable parts (and/or the IOL) therein. In block 1120, the injector 100 is moved from the open position to the closed position. In response to the movement of the injector 100, (for multiple-viewing element IOLs) the viewing elements of the IOL are first longitudinally displaced so that their respective viewing axes are no longer colinear. The displacement of the viewing elements advantageously may be along the longitudinal axis of the injector 100. In response to further movement of the injector 100 to the closed position, the (thus displaced) IOL is then at least partially compacted. The at least partially compacted IOL advantageously may remain on the longitudinal axis of the injector 100. In some embodiments, the injector 100 has a compacted position in which the IOL is at least partially compacted by one or more lens compaction members. A retention force may be applied to at least one of the lens compaction members in order to at least partially retain this compaction member in the compacted position. If the injector 100 comprises an optional automatic plunger lock mechanism, the plunger is automatically unlocked as the injector 100 is moved from the open to the closed position. In block 1130, the plunger is depressed, which displaces the compacted IOL along the longitudinal axis of the injector 100.

Except where otherwise noted, components of the injector 100 may be formed (e.g., via molding) from any suitably rigid material, including plastics such as acrylonitrile butadiene styrene (ABS). In some embodiments, some or all of the injector components may be formed from a transparent plastic such as clear polycarbonate, to promote visibility of the IOL during compaction/delivery. In certain embodiments, components that support and/or displace the IOL (e.g., the support member 212 and/or the lens engagement member 236) may be formed from materials to which the viewing elements 202a, 202b tend to adhere. For example, acetal (available as DELRIN® from DuPont) may be used due to its good adhesion properties with many of the materials (e.g., silicone, polyurethanes, hydrogels, acrylics, PVA, styrene-based copolymers) typically employed to construct IOLs.

It is contemplated that the IOL 200 may be positioned within any of the embodiments of the injector 100 (e.g., with the lens in the storage condition) during manufacture/assembly of the injector 100. The injector 100, with the IOL 200 thus disposed inside, may then be sterilized as a unit, either at the point of manufacture or at some downstream location. Where appropriate, the sterilized injector-lens assembly may be contained in a sterile package, wrapper, bag, envelope, etc. in which the injector-lens assembly may remain until arrival at the point (or time) of use. The injector-lens assembly may be sterilized before and/or after placement in the package. This facilitates a simple point-of-use procedure for medical personnel involved in implanting the IOL 200 contained in the injector 100: after opening (any) packaging, the physician, or other medical personnel, can compact and insert the IOL 200 using the injector 100 as discussed above, without any need for removing the IOL 200 from the injector 100. Accordingly, there is no need to handle the IOL 200 or manually load the IOL 200 into an insertion device at the point of use, both of which can be difficult and tedious, and can compromise the sterility of the IOL.

Although certain preferred embodiments and examples are disclosed herein, inventive subject matter extends beyond the specifically disclosed embodiments to other alternative embodiments and/or uses of the invention, and to modifications and equivalents thereof Thus, the scope of the inventions herein disclosed is not limited by any of the particular embodiments described herein. For example, in any method or process disclosed herein, the acts or operations of the method or process may be performed in any suitable sequence and are not necessarily limited to any particular disclosed sequence. For purposes of contrasting various embodiments with the prior art, certain aspects and advantages of these embodiments are described. Not necessarily all such aspects or advantages are achieved by any particular embodiment. Thus, for example, various embodiments may be carried out in a manner that achieves or optimizes one advantage or group of advantages as taught herein without necessarily achieving other aspects or advantages as may also be taught or suggested herein.

What is claimed is:

1. An injector for an intraocular lens having first and second interconnected viewing elements with respective first and second viewing axes, the injector comprising:
an injector body having a longitudinal axis, the body comprising a first housing and a second housing, the housings configured to move relative to each other in the direction of the longitudinal axis, the first housing configured to receive the lens;
a lens engagement surface configured to engage the first, but not the second, viewing element;
opposing lens compaction members, each comprising a lens compaction surface, the lens compaction members configured to move from a first position in which the lens compaction surfaces are spaced from each other by a distance to a second position in which the lens compaction surfaces are closer to each other than in the first position and in which a lens positioned therebetween is fully compacted, wherein in response to relative longitudinal movement of the housings, the lens engagement surface displaces the first viewing element from the second viewing element in the direction of the longitudinal axis, and the opposing lens compaction surfaces move from the first position to the second position; and
a retention member configured to apply a longitudinal retention force on the lens compaction members to retain the compaction members in the second position.

2. The injector of claim 1, wherein a surface of the second housing comprises the lens engagement surface.

3. The injector of claim 1, wherein a lens engagement member is disposed in the injector body and a surface of the lens engagement member comprises the lens engagement surface, the lens engagement member configured to move longitudinally in response to relative longitudinal movement of the housings.

4. The injector of claim 3, wherein in response to relative longitudinal movement of the housings, the lens engagement member is further configured to move in a direction substantially orthogonal to the longitudinal axis.

5. The injector of claim 1, wherein when the lens is in the first position, the lens is disposed substantially between the lens compaction surfaces.

6. The injector of claim 1, wherein when the lens is in the first position, the lens is disposed substantially along the longitudinal axis.

7. The injector of claim 1, wherein when in the second position, the compaction surfaces are configured to provide a delivery channel for the lens that is substantially coaxial with the longitudinal axis.

8. The injector of claim 7, wherein the injector body further comprises a delivery probe having a passageway along the longitudinal axis, the passageway having a proximal end and a distal end, wherein distal ends of the lens compaction members are configured to mate with the proximal end of the passageway when the lens compaction members are in the second position.

9. The injector of claim 8, wherein the delivery channel has a cross-section that substantially matches a cross-section of the passageway of the delivery probe.

10. The injector of claim 1, wherein the lens engagement surface is configured to displace the first viewing element while the lens compaction members are in the first position.

11. The injector of claim 1, wherein at least one of the lens compaction members comprises a substantially wedge-shaped portion having a distal end having a wedge angle.

12. The injector of claim 11, wherein the wedge angle is in a range from about 10 degrees to about 20 degrees.

13. The injector of claim 11, wherein the wedge angle is about 15 degrees.

14. The injector of claim 1, wherein the retention member comprises an elongated member disposed substantially parallel to the longitudinal axis.

15. The injector of claim 14, wherein when the opposing lens compaction members are in the second position, the elongated member is configured to compress so as to provide the longitudinal retention force.

16. The injector of claim 1, wherein when the lens compaction members are in the second position, the lens compaction surfaces are configured to provide a compaction force on the lens that is in a range from about 1 pound to about 2 pounds.

17. An intraocular lens injector for compacting an intraocular lens having a first viewing element and a second viewing element, the injector comprising:
a housing comprising a first portion and a second portion, the housing providing relative movement between the first and second portions, the housing having a first surface upon which the intraocular lens can be placed in an unstressed condition;
an injection lumen having a projection extending at least partially in the housing, the projection of the injection lumen having a longitudinal axis;
a lens displacement member disposed within the housing opposite of the first surface and movable from a first displacement position relative to the first surface to a second displacement position;
a first lens compacting surface;
at least one movable compacting member comprising a second lens compacting surface disposed opposite the first compacting surface, the movable compacting member having a first compacting position in which at least one of the first and second lens compacting surfaces is spaced away from the projection of the injection lumen and a second compacting position in which the first and second lens compacting surfaces are spaced substantially along the projection of the injection lumen; and
a retention member positioned between the movable compacting member and the housing, the retention member configured to bias the movable compacting member toward the second compacting position.

18. The injector of claim 17, wherein in response to an initial relative movement between the first and second portions, the lens displacement member moves from the first displacement position to the second displacement position, and in response to a further relative movement between the first and second portions, the compacting member moves from the first compacting position to the second compacting position.

19. The injector of claim 17, wherein the initial relative movement and the further relative movement are substantially along the longitudinal axis.

20. The injector of claim 17, wherein the intraocular lens is in the unstressed condition when the lens displacement member is in the first displacement position.

21. The injector of claim 17, wherein the first viewing element is at least partially displaced relative to the second viewing element when the lens displacement member is in the second displacement position.

22. The injector of claim 21, wherein the at least partial displacement of the first viewing element is substantially along the longitudinal axis.

23. An injector for an intraocular lens, the injector comprising:
a delivery lumen extending along a delivery axis;
a lens compactor having a home configuration for retaining the lens in a substantially unstressed condition and a compacted configuration in which the compactor stresses the lens into an at least partially compacted condition, the lens compactor configured to change from the home configuration to the compacted configuration in response to movement of a compactor actuator by a user;
a driving member movable at least partially along the delivery axis and configured to drive the lens along the delivery lumen when the lens is in the at least partially compacted state; and
a locking member having a locked position in which the driving member is substantially restricted from driving the lens when the compactor is in the home position and an unlocked position in which the driving member is substantially unrestricted from driving the lens when the compactor is in the compacted condition, the locking member changing from the locked position to the unlocked position in response to the movement of the compactor actuator by the user.

24. The injector of claim 23, wherein the movement of the compactor actuator is substantially parallel to the delivery axis.

25. The injector of claim 23, wherein the locking member comprises a first portion configured to block movement of the driving member along the delivery axis and a second portion configured to permit movement of the driving member along the delivery axis.

26. The injector of claim 25, wherein the first portion is disposed on the delivery axis when the locking member is in the locked position and the second portion is disposed on the delivery axis when the locking member is in the unlocked position.

27. The injector of claim 23, wherein the intraocular lens comprises first and second interconnected viewing elements, and the lens compactor has a displaced configuration in which the first and the second viewing elements are relatively displaced, the lens compactor configured to change from the home configuration to the displaced configuration and then to the compacted configuration in response to movement of the compactor actuator by the user.

28. The injector of claim 27, wherein the movement of the compactor actuator is substantially parallel to the delivery axis.

29. A method of preparing for implantation an intraocular lens having first and second interconnected viewing elements with respective first and second viewing axes, the method comprising:
providing the intraocular lens along a longitudinal axis of a chamber, the first and the second viewing axes being substantially colinear;
relatively displacing the viewing elements along the longitudinal axis such that the viewing axes are no longer colinear;
moving at least one compaction member to a compacted position in which the lens is at least partially compacted while remaining along the longitudinal axis; and
applying a retention force on the at least one compaction member, to at least partially retain the at least one member in the compacted position.

30. The method of claim 29, wherein the retention force is applied in a direction parallel to the longitudinal axis.

31. The method of claim 29, further comprising automatically unlocking a lens delivery member when the at least one compaction member is in the compacted position, the lens delivery member configured to advance the at least partially compacted lens along the longitudinal axis.

32. The method of claim 29, further comprising advancing the at least partially compacted lens along the longitudinal axis while the at least one compaction member is in the compacted position.
